(19) United States
(12) Patent Application Publication
(10) Pub. No.: US 2023/0039224 A1
(43) Pub. Date: Feb. 9, 2023

(54) PYROLYSIS OIL FROM RECYCLED POLYMER HAVING INCREASED OLEFINS AND DECREASED CHLORIDES AND METALS

(71) Applicant: UOP LLC, Des Plaines, IL (US)

(72) Inventors: Andrea G. Bozzano, Northbrook, IL (US); Christopher James Anderle, Roselle, IL (US); Gregory William Fichtl, Chicago, IL (US); Ping Sun, Hinsdale, IL (US)

(21) Appl. No.: 17/858,007

(22) Filed: Jul. 5, 2022

Related U.S. Application Data
(60) Provisional application No. 63/222,157, filed on Jul. 15, 2021.

(51) Int. Cl. C10G 1/10

(52) U.S. Cl. CPC C10G 1/10; C10G 2300/1003; C10G 2300/201; C10G 2400/22; C10G 2400/30

(57) ABSTRACT

A pyrolysis oil is produced that has a low level of contaminants such as chlorides and metals. The process that is used is without the use of a hydrotreater but instead has both a pretreatment section to target polyvinyl chloride as well as non-plastics including metals and a secondary chloride removal step to first melt the plastic and remove evolved HCl gas. Adsorbents are used to polish the chloride and metal content to an acceptable level. The pyrolysis oil has a significant olefins content such as 36-56 wt %.

### CROSS-REFERENCE TO RELATED APPLICATIONS

This application claims priority from U.S. Provisional Application No. 63/222,157, filed Jul. 15, 2021, which is incorporated herein in its entirety.

### FIELD

The field of the invention is the production of olefin-rich, low impurity pyrolysis oil from recycled polymers. More particularly, the invention relates to the production of the pyrolysis oil without the use of a hydrotreating step to remove contaminants.

This invention results in recycled polymer pyrolysis oil (RPPO) that is fit for use in downstream upgrading units (steam crackers, other petrochemicals or refinery units) in terms of key contaminant content, such as reduction in level of chloride and metals, and retaining olefins content without the need to hydrotreat the RPPO to align with the feed and contaminant requirements of the downstream unit.

### BACKGROUND OF THE INVENTION

Mixed plastic waste mostly originates from curbside waste collection of post-consumer plastic waste. Mixed plastic waste also comes from specific industrial sites e.g., construction, packaging and agricultural wastes that have a broad range of compositions. A waste recycling facility applies a range of sorting steps to recover recyclable plastics. Some valuable plastic types are recovered as feed for mechanical recycling. The rest of the recovered mixed plastic stream typically contains a range of non-plastic contaminants, such as paper, metal, dirt and organic waste. The plastic stream recovered may contain polyvinylchloride or other halogen containing species from polymerization processes.

Due to some hard to remove contaminants, the recovered mixed plastic stream may not be suitable to be used as a feed to mechanical recycle and is frequently sent to landfill or incineration. Chemical recycling, such as a pyrolysis process, is suitable to further convert the end-of-life plastic waste stream to a fuel or more preferably a petrochemical feedstock to make monomers to sustain recycle circularity for polymers. A pyrolysis process is typically done in an air-free atmosphere and ay higher temperature conditions, e.g. 350° C. to 900° C. European patent 3186556B1 discloses a pyrolysis apparatus with a mixer and contactor. There are a number of examples of plastic pyrolysis.

Raw pyrolysis oil typically contains paraffins, olefins and aromatic hydrocarbons. The impact of feed composition on pyrolysis oil hydrocarbon types has been a focus of many research studies and have been widely understood through detailed analytical characterizations. Pyrolysis of a waste polymer frequently produces a range of olefinic hydrocarbon due to chains of free radical reaction including a typical carbon-carbon bond breaking due to unpaired electrons or a radical that moves freely cross sites. This is known as beta scission that results in an olefin molecule and a new radical that continues the cracking reaction. The significance of this phenomena is that olefins are commonly among pyrolysis oil products especially when the waste polymer contains polypropylene and polyethene.

Typically, raw pyrolysis oil contains heteroatom molecules. The most common contaminants are halogens. Polyvinylchloride (PVC) frequently is found in a mix of waste polymers. Complete mechanical separation of PVC is unrealistic even through the use of the most sophisticated technology. In addition, halogen-containing chemicals are commonly used as flame retardants in polymerization process. As a result, halogens including chlorine, bromine and fluorine are commonly found in pyrolysis products since not all halogens can be discharged through either unpyrolyzed residue or an incondensable hydrogen halogen gas. compound in gas phase. Halogens contained in pyrolysis oil lower the oil's value in downstream processing, e.g. in a steam cracker that uses a waste polymer derived pyrolysis oil as feedstock. For example, the metallurgy can be seriously damaged when chloride level exceeds limits for reactor metallurgy. In addition, other contaminants are likely, for example trace metals and silicon.

Plastic circularity requires producing pyrolysis oil of acceptable quality out of waste plastic. Chlorides in pyrolysis oil are known to cause metallurgy issues due to stress corrosion cracking. Most pyrolysis technologies for plastics apply a hydrotreating step to prepare the pyrolysis oil for upgrading in downstream units as a clean-up step hydrodechlorinating and hydrogenating olefins. The resulting pyrolysis oil is similar to conventional petroleum oil products after treatment.

The present invention is to produce a novel pyrolysis oil composition directly as a feedstock to downstream process units without hydrotreating. The pyrolysis oil, only having undergone very light treatment to remove trace contaminants, is largely unaltered in terms of hydrocarbon species relative to a pyrolysis oil that has been hydrotreated. As such, the pyrolysis oil subject to this invention contains significant amounts of olefins, but very low levels of other trace contaminants like halogens and metals. Other pyrolysis oils that have been hydrotreated would have their olefins saturated to make paraffins, such that the composition is distinct from the pyrolysis oil that is the subject of this invention. The inventors' laboratory results show olefins that are mostly mono olefin, which behave similarly to paraffins at downstream processing steps. The pyrolysis oil is deeply cleaned of halogens and trace metals. This pyrolysis oil is suitable for being used as a feedstock directly to downstream petrochemicals or refining process units, including but not limited to fluid catalytic cracking (FCC) unit or a steam cracking unit.

From a process design viewpoint, preparing RPPO with properties suitable for downstream process units does not require a hydrotreater, but can instead be accomplished with various processing steps to produce an RPPO with sufficiently low chloride and metals content to meet feed composition requirements in the downstream process units. The pyrolysis oil is only processed as much as needed to produce molecules that are used to produce plastic products in the circular economy.

The business advantage is that the number of processing steps are reduced, resulting in a more efficient preparation of RPPO in terms of both capital and operating expenses, yielding a competitive advantage for the producer relative to other pyrolysis oils. For example, a typical hydrotreating unit sized for recycling scales can cost on the order of $50-100 million and requires a supply of hydrogen with operations at elevated temperatures and pressures, requiring significant capital and operating expenses, nearly all of which is avoided herein.

### SUMMARY OF THE INVENTION

The ready-to-use pyrolysis oil consists of paraffins, olefins and aromatics as produced from the pyrolysis unit. The hydrocarbon species that make up the pyrolysis oil subject to this invention are largely left intact after the pyrolysis reaction step, preserving the olefins, paraffins, and other hydrocarbon species naturally produced in pyrolysis reactions. Excessive clean-up steps of RPPO, such as hydrotreatment, is avoided, reducing processing costs and capital expense. Pyrolysis oil normally contains trace amounts of halogens and trace metals at ppm levels even after clean-up steps are taken. In comparison, the ready-to-use pyrolysis oil subject to this invention is best described as paraffin and olefin-rich with low contaminants, such as halogens and trace metals. The ready-to-use pyrolysis oil is a final product from a pyrolysis plant that is directly applied as a feedstock to downstream petrochemical or refining process units. The pyrolysis oil composition is novel due to surprising laboratory testing results showing that there is a paraffin and olefin-rich oil with very low contaminant levels, and which behaves comparably to conventional petroleum-derived feedstocks in terms of main product yield in FCC or stream cracking units, among other process units.

There may be a number of ways to achieve the objective of producing a ready-to-use pyrolysis oil. One method described in this disclosure is through a sequence of process steps. A pretreatment is needed to treat non-plastic contaminants (including some metal, paper, wood and rubber) to reasonably low levels. At the pretreatment step, oxygen-containing plastic is considered as a main contaminant, such as polyethylene terephthalate (PET). PVC is also a target for pre-sorting however, complete elimination of PVC is not necessary because multiple process steps downstream within the pyrolysis unit can also help increase the efficiency of chloride removal.

The current process may include a chloride removal step via controlling melting temperatures of the plastic feed before it is introduced to the pyrolysis reactor. In the melt tank, hydrogen chloride (HCl) is evolved and removed from the system, further reducing chloride content of the mixed plastics. After processing the mixed plastics stream through the pyrolysis reactor, the effluent stream is treated to remove chloride and trace metals through a reaction or a removal step to acceptable levels for downstream petrochemical or refining process units, such as steam cracker or FCC process units. The current process accomplishes low metal and chloride levels without hydrotreating, saving millions of dollars in capital and operations expenses relative to competitive waste plastics pyrolysis processes. Acceptable levels for chloride and trace metals are in a few parts per million, but at most less than 10 ppm.

In another aspect of the invention, the pyrolysis oil has properties that have not been previously attainable for a pyrolysis oil in that the RPPO has a high olefin content at the same time it has very low content of other contaminants. In particular, there are at least more than 20 wt % olefins, less than 10 ppmw chlorides and less than 10 ppmw inorganic molecules. In a preferred embodiment, there are 36-56 wt % olefins, 17-28 wt % paraffins and about 14-20 wt % aromatics.

In another embodiment the novel pyrolysis oil composition is provided by a process that takes a mixed plastic waste stream comprising PVC and other plastic materials. This process comprises sending the mixed plastic stream through a pretreatment step to partially remove any PET and PVC partially, shred the plastics in the plastic waste stream, and then melting the plastics in a melting reactor at a sufficient temperature to produce a liquid mixture of plastics, The liquid mixture of plastics is sent to a pyrolysis reactor to produce a pyrolysis oil in a condensate format. Then the pyrolysis oil may be sent through a fixed bed reactor to remove chlorides and chloride-containing materials by a solid adsorbent or a reactant in a non-hydrogen environment to produce a clean pyrolysis oil stream. While any adsorbent that is effective in removing chloride species may be used, the solid adsorbent may consist of one or more active metal species impregnated in a silicate alumina support that selectively absorb or react with chloride species but stay inert to hydrocarbon molecules in pyrolysis oil including olefinic molecules. Residual chloride level is reduced to 10 ppmw, inorganic molecules down to 10 ppmw and olefin content >20% wt. The pyrolysis oil consists of 20-80% wt olefinic molecules as a function of the plastic waste stream composition. The clean pyrolysis oil maintains a minimal conversion of olefin. In an embodiment, the mixed plastic stream has up to about 10 wt % PVC in the mixed plastic stream and up to 2% wt PVC in the liquid mixture of the plastics. The clean pyrolysis oil stream may be used in a fluidized catalytic cracking (FCC) process or a steam cracking process as a blend component, or a combination with streams from other process technologies involving FCC, steam cracking with other process technologies at an end user determined location, or several other downstream processing possibilities that may or may not include FCC or stream cracking. An important advantage of the invention is the production of a clean pyrolysis oil consisting of metal elements less than 10 ppmw of combined metal species including calcium, silicon, aluminum, iron and a range of others. The melting reactor is operated at a temperature from about 280° C. (536° F.) to about 330° C. (626° F.), the pyrolysis reactor is operated at a temperature from about 380° C. (716° F.) to about 450° C. (842° F.) and the adsorbent bed is operated at a temperature from about 100° C. (212° F.) to about 300° C. (482° F.). The mixed plastic stream may have up to about 10 wt % PVC from the feed supply and up to 3% wt in the liquid mixture.

### Definitions

As used herein, the term “reactor” means a thermal cracking vessel that provides residence time for feed polymers. The melting tank reactor is a reactor where only a portion of a mixed plastic feed is pyrolyzed when the majority of the mixed plastic feed goes through physical melting into a viscous liquid. The main pyrolysis reactor types are introduced above, a well-mixed reactor type of using convective heat transfer has advantages over indirectly conductivity heater transfer offered by a kiln or a screw extruder. Well-mixed reactor sees uniform temperature distribution established throughout the liquid space.

As used herein, the term “mixed plastic feed” means two or more polymers are present in the feed.

As used herein, the term “product” means a portion of mass stream, after the pyrolysis reaction. A product can be broad as an intermediate product that require further processing and a finished product that is collected at the very end of the process that has a marketable value that may be sold for profit. This is in contrast with a byproduct when aiming for the main profitable product. In the current context, the pyrolysis reaction produces residue gaseous product containing a hydrocarbon gas, in 5-10% wt of the melt feed, a liquid when condensed to room condition in 70-90 wt % of yield, 2-15% wt of a residue that leaves from the reactor discharge as a mix of liquid and solid. The hydrocarbon gas and residue are byproducts. The liquid collected when condensed is an intermediate product that requires further treatment. The liquid product after treatment that is free of contaminants and have marketable value to downstream customers is final product.

As used herein, the term “residue” means a portion remaining after a process step. In the current context, a residue is specifically a stream that leaves the process boundary as a mix of liquid and solid that has relatively lower profitable use to downstream applications than the main product. In this context, a residue byproduct can contain char and inorganic residue. A char is a necessary byproduct when making main product. A reaction strategy may be applied to reduce char, but it cannot be eliminated. Certain plastic compositions contribute to yielding char in higher amount than another. It is known that rigid plastic and aromatic molecule containing plastic compounds, such as PVC, PET, PS or acrylonitrile butadiene styrene from electronic waste tend to make more char than polyethylene and polypropylene at comparable processing conditions. Inorganic residues originate from layered additives introduced during polymer manufacturing processes. One example is MgO, CaO and Li2O based glass fiber species. Another example is zinc, lead or cadmium based metallic fillers when forming conductive plastics. Metal or alkali metal ends up in the residue stream in a solid format.

As used herein, the term “quality” pyrolysis oil product quality refers to many chemical compositions that make it more or less suitable to a downstream application. A common objective of mixed plastic pyrolysis is to create a product that can be used in downstream customer plants such as a refinery or a petrochemical plant. The hydrocarbon content is important measure of quality of pyrolysis oil. In particular, a key quality measure relevant herein is the halogen content. Out of the halogen elements, chloride ions are the most concerning. The chloride content, either in organic or inorganic format, tends to lead to metallurgy corrosion. Another key quality measure is the amount of trace metals. Trace metals from layered additives introduced during polymer manufacturing processes find fates mostly in reactor residue, a very small fraction of them ends in raw pyrolysis oil product. A deep-cleaning is needed to remove trace metals down to ppm levels.

As used herein, the term “portion” means an amount or part taken or separated from a main stream without any change in the composition as compared to the main stream. Further, it also includes splitting the taken or separated portion into multiple portions where each portion retains the same composition as compared to the main stream.

As used herein, the term “unit” can refer to an area including one or more equipment items and/or one or more sub-units. Equipment items can include one or more reactors or reactor vessels, heaters, separators, drums, exchangers, pipes, pumps, compressors, and controllers. Additionally, an equipment item, such as a reactor, dryer, or vessel, can further include one or more units or sub-units.

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” or “directly” means that flow from the upstream component enters the downstream component without undergoing a compositional change due to physical fractionation or chemical conversion.

### DETAILED DESCRIPTION

The following detailed description is merely exemplary in nature and is not intended to limit the various embodiments or the application and uses thereof. Furthermore, there is no intention to be bound by any theory presented in the preceding background or the following detailed description.

Pyrolysis oil can be efficiently cleaned using non hydrogeneration/non-hydrotreating process to produce a suitable feedstock that feeds directly to downstream process units. Surprisingly, the pyrolysis oil compositions that were obtained prove that the oil serves as an efficient feed to generate good yield and competitive quality in downstream process units.

Pyrolysis oil from mixed waste plastic is generally well known. When the quality of a waste plastic stream is not enough for conventional mechanical recycling, it can be fed preferably into a pyrolysis process. The process chemistry of pyrolysis is well known to go through a chain of free radical cracking reactions forming a broad range of hydrocarbon compounds. Free radical cracking reactions are complex but fall into a few basic categories, such as radical initiation, beta scission, hydrogen transfer through either intramolecular or extramolecular route depending on hydrogen availability and termination reactions.

The product compositions from pyrolysis of major polymers are relatively well understood. Polyethylene goes through random initiation and tends to form olefins out of a series of beta scission reactions. The hydrogen availability can be a combination of intramolecular or extramolecular, thus the product frequently contains a broad range of carbon numbers, with majority being parrafins, followed by olefins and some aromatic compounds. Formation of olefins is intrinsic to free radical cracking when carbon-carbon bond breaking due to radicals leads to an olefin and another new radical. A small amount aromatic compounds form in cases where there is a lack of hydrogen availability, resulting in aromatization as a means to form a thermodynamically stable product. Polypropylene goes through pyrolysis reactions similarly but has a unique difference. Radical electrons travel along tertiary carbon locations while abstracting hydrogen internal to molecules, frequently forming propylene oligomers with a variety of carbon lengths but tends to be less continuous in terms of carbon number distribution. As a result, polypropylene pyrolysis forms propylene oligomers mostly with one double bond. The backbone of olefins features either continuous broad range of carbon distribution with less branching from polyethylene or with evenly spaced carbon numbers in multiples of 3 as propylene oligomers from polypropylene pyrolysis. However, the similarity is that olefins derived from either polyethylene or polypropylene polymers are rich in mono-olefins with one double bond at the end of a chain at the alpha location.

PVC pyrolysis starts from the C—Cl bond, hydrogen transfer mostly through abstracting internal hydrogen forming gaseous HCl. When a mixture of pyrolysis oil product travels to downstream reactors, HCl is likely to react with olefins and form alkyl chloride. A small portion of HCl may also carry downstream due to its solubility in the pyrolysis oil. Inorganic HCl and organochlorides are considered contaminants in pyrolysis oil.

Other major polymers such as polystyrene and PET are also known to produce styrene oligomers and naphthenic acid respectively.

Contemporary plastic commodities have a range of additives for enhancing polymer properties. Mica powder and calcium carbonate are commonly known additives to enhance mechanical properties. Some halogens, including PVC itself, or fluorine and bromine are common flame retarding additives. Other additives serve as a range of functionality, such as photo stability, antioxidant and heat stability, among other applications. Such additives may end up as contaminants in the pyrolysis oil product. When Chlorine, Fluorine, Bromine and trace metal are contained in pyrolysis oil, there is a need to clean remove them to sufficiently low levels.

Many disclosures in the waste polymer pyrolysis field focus on process and specialty apparatus only, examples are EP3186556B1, US20140114098A1 and US20170283706A1. Raw pyrolysis oil cannot be directly used due to its underlying quality issues. There are fewer concerns for customers to use raw pyrolysis oil as a replacement fuel in a combustion or a burner that has less stringent requirements for contaminants. However, oil refinery or petrochemical plant owner-operators require more strict limits on chloride and trace metals. High levels of chlorides in pyrolysis oil can prevent downstream processes from using it as a feedstock. Trace metals in a pyrolysis oil sent downstream either are removed in a downstream process via targeted contaminant removal, such as metal guard bed catalysts, or possibly metals can pass through all downstream processes and end up in finalproducts.

Some refining and petrochemical processes categorize olefins as a contaminant. Double bonds internal to a molecule are known to be involved in complex reactions leading to low conversion to end products and side reactions leading to coke formation. However, a single double bond, and especially at the alpha carbon location, tend to crack similarly to paraffins of similar carbon number. The double bond at alpha carbon location can contribute directly to yield of end products of a cracking process, such as FCC or steam cracking, among other possible downstream processes.

Most common raw pyrolysis oil treatment is done by hydrotreating. Under mild hydrotreating conditions, both hydrodechlorination and olefin saturation occur. Hydrodechlorination is well known and capable of achieving close to complete removal of halogens, e.g. organohalogens through hydrogenation mechanism. According to this disclosure, hydrotreating conditions remove halogens and olefins simultaneously. Many pyrolysis technologies are practiced applying a hydroprocessing process downstream of the pyrolysis unit. There is significant, if not complete, saturation of olefins when chloride is removed.

This invention teaches that after the waste plastic pyrolysis process, the olefin content of the oil effluent needs to be preserved, with minimal olefin saturation or other removal of the olefins. In addition, the pyrolysis oil needs to be cleaned down to 0-10 ppm w of chloride and preferably 0-5 ppmw of total trace metals to be a unique composition to be used by a range of downstream processes.

Olefinic molecules from waste polymer-derived pyrolysis oil are mainly mono- and alpha-olefins. Further removal of olefins from raw pyrolysis oil, particularly by expensive processes such as hydroprocessing, does not make economic sense and should be avoided.

For the purposes of demonstrating the advantages of the composition produced herein, two examples are provided of downstream pyrolysis oil upgrading by FCC and steam cracking, and how their feed specifications require careful management of contaminants levels in the pyrolysis oil to maximize pyrolysis oil's value as a feedstock.

Fluid catalytical cracking (FCC) applies high cracking temperatures and acidic solid catalysts to petroleum-based feedstock, such as a vacuum gas oil, a coker gas oil or a residue oil. FCC has good selectivity to main products of gasoline and propylene, as well as side products such as a light cycle oil. When propylene is collected, it becomes a building block for polypropylene. When an alpha-olefin rich pyrolysis oil is cracked at FCC conditions, it has been found to provide surprisingly good results as compared with regular FCC feedstock, particularly in terms of propylene yield. FCC units have low tolerance for chloride and trace metal content, such as <˜2 ppm on total feed oil or <˜10 ppm of a feed blending component. Deep cleaning of chloride and trace metal in any feed blending component, including waste plastics pyroysis oil, is required.

The steam cracking (SC) process is commonly used to process a naphtha feed for producing a range of light olefins and butadiene products for petrochemical manufacturing. Ethylene is a particularly high value product that is a building block for polyethylene. The SC unit has low tolerance for chloride and trace metal content, such as <˜1 ppm on total oil or <˜10 ppm on a feed blending component. Deep cleaning on chloride and trace metals is required. A technical paper published at the AICHE Spring Meeting 2020 showed waste plastic-derived raw pyrolysis oil without any clean-up produces comparable ethylene yield as conventional light naphtha at identical pilot plant conditions. According to this disclosure, olefins in pyrolysis oil is best preserved, while halogens and trace metals are best removed to sufficiently low level. Olefins along with paraffins in pyrolysis oil derived from waste plastics, such as waste polypropylene and polyethene, are highly valuable feedstocks.

According to this disclosure, the finished pyrolysis oil composition feeding to typical downstream processes including FCC, steam cracking, and others, may be a mixture of olefin, paraffin and aromatic molecules as produced from raw pyrolysis process with <10% wt of mass disappearance due to the post-pyrolysis step in each class relative to its mass content in raw pyrolysis oil, or preferably <2% wt of mass disappearance. The finished pyrolysis oil is treated downstream of the pyrolysis reactor such that halogen and trace metals levels are each down to <10 ppm. As far as the inventors know, the pyrolysis oil composition subject to this invention and its direct use in downstream process units has not been taught in any of the prior art. The processes to produce a pyrolysis oil with the composition as described herein may vary in many details. This disclosure only teaches one process that may produce oil composition as described. The methods to produce the subject invention's oil composition may go beyond the processes taught herein.

The first step involves pretreatment of a mixed plastic stream. As described above, non-plastic contaminants contribute to production of a carbon-rich, hydrogen-deficient residue, and may result in operational difficulties in the pyrolysis process. Thus, a pretreatment process may be designed to eliminate non-plastic contaminants as much as possible. Among the non-plastic contaminants, paper and cardboard are commonly removed using an air separation technique which uses a stream of rapidly moving air to eject such contaminants from the passing waste stream. Ferrous metals are taken out by magnet, and non-ferrous metal, such as aluminum cans, can be taken out by electrical current force through an eddy current device.

For further sorting of plastic waste, modern sorting technology has advanced to a level that targeted selection or rejection may be achieved through “fingerprint” sorters designed to identify individual polymer types and sort them accordingly. Near infrared sortation detects polymer type at the plastic surface and positively picks or negatively rejects using compressed air jets to lift plastics off a conveyer belt. Electromagnetic and robot sortation is used for more advanced sorting. There are also sorting techniques that mainly target PET, such as manual processes that use ultraviolet light that shows PET as appearing blue while PVC items will be yellow or green when exposed to UV light. It is important to remove PET because oxygenates formed from PET pyrolysis have low value, cause operational issues in the pyrolysis process, and cause the product pyrolysis oil to have weaker acidity than desired. Bulk PVC is removed from sorting step.

A sorted plastics-rich stream may be processed in a 2-stage pyrolysis process, with the first reactor being a melt reactor and the second reactor being the main reactor. The first step targets at low temperature window for selective de-halogenation in the melt reactor. PVC dechlorination and other dehalogenation occurs at a much lower temperature than other polymer decomposition. The first stage melt reactor pyrolysis reactions may be achieved with a range of reactor types, such as a reactor with a screw feeder, a rotary kiln reactor, a reactor with mixing by a stirring or by liquid momentum. Plastics pyrolysis reactions are endothermic, so all reaction heat must be provided by a burner, an exchanger, or a heat-integrated process flow that provides heat input. Dehalogenation conditions may be in a temperature range, for example, from about 270° C. (518° F.) to about 350° C. (662° F.), or preferably about 300° C. (572° F.) to about 330° C. (627° F.). Pressure, residence time, and nitrogen sweeping rate of the first stage melt reactor impacts the gaseous hydrogen-halogen disengagement. For example, the first stage melt reactor may run at a pressure from 0.069 MPa (gauge) (10 psig) to about 1.38 MPa (gauge) (200 psig) and a liquid hourly space velocity from about 0.1 hr⁻¹ to about 2 hr⁻¹ and is operated under a nitrogen blanket at a dedicated nitrogen sweeping rate of about 1.7 Nm³/m³ (10 scf/bbl) to about 170 Nm³/m³ of plastic melt (1,000 scf/bbl). The first step melt reactor process produces two product streams, an overhead gaseous stream enriched in halogens from dechlorination and other dehalogenation reactions, and a liquid stream containing mainly melted plastic.

The second stage of pyrolysis increases the severity of the conditions such that the melted plastics coming from the first stage melt reactor are thermally pyrolyzed. The second stage pyrolysis may apply a range of reactor types, such as a reactor with a screw feeder, a rotary kiln reactor, a reactor with mixing by stirring or by liquid momentum. Plastics pyrolysis reactions are endothermic, so all reaction heat must be provided by a burner, an exchanger or a heat-integrated process flow that provides heat input. Second stage pyrolysis conditions may operate at a temperature from about 300° C. (572° F.) to about 550° C. (1022° F.), or preferably about 380° C. (716° F.) to about 450° C. (842° F.), a pressure from about 0.069 MPa (gauge) (10 psig) to about 1.38 MPa (gauge) (200 psig), or preferably about 0.138 MPa (gauge) (20 psig) to about 0.345 MPa (gauge) (50 psig), a liquid hourly space velocity of the fresh melt feed from about 0.1 hr⁻¹ to about 2 hr⁻¹, or from about 0.2 hr⁻¹ to about 0.5 hr⁻¹. The second stage process produces two product streams, an overhead gaseous stream containing a vapor product enriched in hydrocarbon species resulting from the pyrolysis of the incoming melted plastics, and a liquid stream that is generally characterized as a residue containing unconverted polymer, inorganic metals and carbonaceous coke.

The vapor product from the main pyrolysis reactor is the main product stream and requires multiple condensing steps through a collection of exchangers between hot and cold process streams, quench tower with cooling medium, or a range of cooling and separation unit operations including fractionation towers, stripping towers or non-direct contact air cooling. The purpose of condensing is to recover the main liquid product stream that separates from non-condensible gas byproduct upon cooling. The non-condensible gas product contains light hydrocarbon species, typically with less than 5 carbon atoms. There may be some residual unconverted halogens remaining in the gaseous product stream from the main pyrolysis reactor. Hydrogen-halogen species formed in second stage main reactor will be concentrated in the non-condensible gas product stream. The entire non-condensible stream is either sent to an oxidizer to be converted to non-hazardous effluent streams or burned for internal energy recovery.

The main liquid condensate is the raw pyrolysis product, containing olefin, parrafins, and aromatic molecules. It may contain also small residual amounts of oxygen-containing molecules, even though PET treatment at the front end of the process should have already removed most oxygen-containing polymer from the system. Olefins are typical products of plastics pyrolysis, depending on the composition of mixed waste plastics to the pyrolysis unit. Polypropylene in the mixed waste plastic feed produces higher olefin content in the raw pyrolysis oil condensate than polyethylene. Polyethylene yields ˜20-50% wt olefins, while polypropylene yields 40-90% wt olefins. Olefins from both polyethylene and polypropylene contain mostly one double bond, namely mono-olefins. The double bond frequently, if not always, is located at the alpha carbon location. Diolefins are rare and present only in trace amounts. Olefins are easily hydrogenated under typical hydrotreating conditions known to someone experienced in the art. Olefins in raw pyrolysis oil condensate may be best preserved by not contacting hydrogen or hydrotreating catalysts. Hydrotreating conditions may include a reaction temperature from 66° C. (151° F.) to about 426° C. (800° F.), or about 316° C. (600° F.) to about 418° C. (785° F.) or about 343° C. (650° F.) to about 399° C. (750° F.) and a hydrogen partial pressure from about 1.4 MPa (gauge) (200 psig) to about 8.2 MPa (gauge) (1200 psig) and a liquid hourly space velocity from about 0.25 l/hr (gauge) to 10 l/hr. Suitable hydrogeneration catalysts are typically comprised of at least one Group VIII metal, or iron, cobalt and nickel, or nickel and/or cobalt and at least one Group VI metal, such as molybdenum and tungsten, on a high surface area support material, such as alumina. Preservation of olefins in pyrolysis oil are a key teaching of this disclosure.

According to this disclosure, a light post-treatment purification step—one not involving hydrotreatment or hydrogenation—is applied by contacting the freshly produced raw pyrolysis oil condensate with a reactive sorbent or non-reactive adsorbent such that halogen contaminants are removed from the pyrolysis oil. According to this disclosure, the suitable solids are ideally basic solids, such alkaline oxides that contains oxygen ions. Alkaline earth oxides react or chemisorb chloride species, likely through a decomposition of organochloride first, followed by reacting or chemisorbing hydrogen chloride subsequently. Alkaline earth oxides can be MgO, CaO, BaO and NaO etc. Other metal oxides, such as FeO, CuO, NiO and MnO may also has similar functionality.

Halogen purification is preferably done by a fixed bed set-up. Halogen is contained using basic solid, spent agents are sent for final disposal. More preferably the purification system has lead-lag capability, e.g. when one train of the purification unit is spent, it is turned off and disposed when another train of the unit is brought to stream.

However, according to this disclosure, in a practical sense, not all feedstock quality, e.g., chloride content in feed to pyrolysis, can be processed by contacting any one of the basic solid agents listed above to achieve the quality required as feed to downstream process unit. Regarding the quality required for downstream units it is typically required that halogens and trace metals each be less than 10 ppm. In cases when chloride in pyrolysis oil exceeds 1-2% wt, a tremendously high degree of de-halogenation needs to be achieved.

According to this disclosure, not all naturally occurring halogen and metals removal agents work to the same extent due to capacity requirement. An activated halogen and metals removal agent on a proper support is likely needed to minimize the volume or size of purification reactors. An activated purification agent is more preferably impregnated to a dispersed support. The dispersed support provides enough surface area to allow maximize use of the purification agent. According to this disclosure, the capacity of halogen retention needs to be greater than a certain threshold to be economically feasible.

As previously explained, the primary objective of the purification step is to reduce halogens in the pyrolysis oil to acceptable levels. However, proper design of the purification reactor can result in removal of trace metals out of pyrolysis oil, in addition to halogen removal. The trace metals, with examples shown above, such as mica (SiO₂), or CaCO₃, or Fe₂O₃, among others, are mostly added in form of finely ground inorganics. Their existence in pyrolysis oil is also in an inorganic format. According to this disclosure, the inorganic nature of the metals present in pyrolysis oil eliminates the needs for hydrodemetalation. This is in contrast to demetallation of petroleum-derived oils, where metals mostly exist in an organic state, such as metalloporphyrin structure. The same purification bed with engineered bed loading design used for halogen removal works surprisingly well for removing trace inorganic metal out of pyrolysis oil, again without the need for hydrotreating.

### EXAMPLE

A pyrolysis oil product example is given below.

### Example 1. Recycled Plastic Derived Pyrolysis Oil Composition

| | Final product |
|---|---|
| Feed type | Adsorbent bed effluent product |
| IBP/50%/T95/FBP, ° C., D2887 | 35/190/358/477 |
| DENSITY, 15.6, g/cc | 0.7769 |
| 150° C.-, 150-380° C., 380° C.+ | 37/61/3 |
| C/H/N/S PPM/O | 84.2³/14.1³/0.16³/18 ppm³/0.05 |
| CL, ppmw | 1-3 |
| Bromine Number | 72 |
| Trace metals, ppmw | 2 |
| Parraffin/Olefin/Aromatics | 47/45/8 |
| Flash point, ° C. | 1-3 |

### Specific Embodiments

While the following is described in conjunction with specific embodiments, it will be understood that this description is intended to illustrate and not limit the scope of the preceding description and the appended claims.

A first embodiment of the invention is a pyrolysis oil composition comprising greater than 20 wt % olefins and between about 0.5 to 20 ppm halogens by weight and a total trace element content between about 0.5 to 15 ppm by weight. An embodiment of the invention is one, any or all of prior embodiments in this paragraph up through the first embodiment in this paragraph wherein the pyrolysis oil composition is further comprising a range of paraffin compounds and a smaller fraction of aromatics. An embodiment of the invention is one, any or all of prior embodiments in this paragraph up through the first embodiment in this paragraph wherein the pyrolysis oil composition is comprising 20-80 wt % of said olefins. An embodiment of the invention is one, any or all of prior embodiments in this paragraph up through the first embodiment in this paragraph wherein the pyrolysis oil composition is suitable for use without further treatment in a refinery or a petrochemical process. An embodiment of the invention is one, any or all of prior embodiments in this paragraph up through the first embodiment in this paragraph wherein the pyrolysis oil composition is suitable for use without further treatment in combination with a refinery or a petrochemical process. An embodiment of the invention is one, any or all of prior embodiments in this paragraph up through the first embodiment in this paragraph wherein the trace elements are selected from calcium, silicon, aluminum and iron. An embodiment of the invention is one, any or all of prior embodiments in this paragraph up through the first embodiment in this paragraph wherein the halogens comprise chloride, fluoride and bromide containing molecules. An embodiment of the invention is one, any or all of prior embodiments in this paragraph up through the first embodiment in this paragraph wherein the pyrolysis oil composition is comprising about 36-56 wt % olefins. An embodiment of the invention is one, any or all of prior embodiments in this paragraph up through the first embodiment in this paragraph wherein the pyrolysis oil composition of claim 1 is comprising about 17-45 wt % paraffins. An embodiment of the invention is one, any or all of prior embodiments in this paragraph up through the first embodiment in this paragraph wherein the pyrolysis oil composition is comprising about 14-20 wt % aromatics. An embodiment of the invention is one, any or all of prior embodiments in this paragraph up through the first embodiment in this paragraph wherein the pyrolysis oil composition is comprising about 0.5-10 ppm by weight chloride. An embodiment of the invention is one, any or all of prior embodiments in this paragraph up through the first embodiment in this paragraph wherein the pyrolysis oil composition is comprising about 0.5-10 ppm by weight of said total trace metal elements. An embodiment of the invention is one, any or all of prior embodiments in this paragraph up through the first embodiment in this paragraph wherein the pyrolysis oil composition is derived from a recycled plastics composition comprising from about 0.5-10 wt % polyvinyl chloride. An embodiment of the invention is one, any or all of prior embodiments in this paragraph up through the first embodiment in this paragraph wherein the recycled plastics composition comprises about 0.2 to 2 wt % polyvinyl chloride.

A second embodiment of the invention is a process of making a pyrolysis oil composition from a recycled plastics feed stream comprising sending said recycled plastics feed stream to a pyrolysis process to produce said pyrolysis oil composition comprising greater than 20 wt % olefins and between about 0.5 to 20 ppm halogens and a total trace element between about 0.5 to 10 ppm. An embodiment of the invention is one, any or all of prior embodiments in this paragraph up through the second embodiment in this paragraph wherein the pyrolysis oil composition is suitable for use as a feed without further treatment in a refinery or a petrochemical reactor. An embodiment of the invention is one, any or all of prior embodiments in this paragraph up through the second embodiment in this paragraph wherein the pyrolysis oil comprises about 36-56 wt % olefins about 17-40 wt % paraffins and about 14-20 wt % aromatics. An embodiment of the invention is one, any or all of prior embodiments in this paragraph up through the second embodiment in this paragraph wherein the trace elements are selected from calcium, silicon, aluminum and iron. An embodiment of the invention is one, any or all of prior embodiments in this paragraph up through the second embodiment in this paragraph wherein the halogens comprise chloride, fluoride and bromide containing molecules. An embodiment of the invention is one, any or all of prior embodiments in this paragraph up through the second embodiment in this paragraph wherein the recycled plastics feed stream comprises about 0.2 to 10 wt % polyvinyl chloride.

1. A pyrolysis oil composition comprising greater than 20 wt % olefins and between about 0.5 to 20 ppm halogens by weight and a total trace element content between about 0.5 to 15 ppm by weight.

2. The pyrolysis oil composition of claim 1 further comprising a range of paraffin compounds and a smaller fraction of aromatics.

3. The pyrolysis oil composition of claim 1 comprising 20-80 wt % of said olefins.

4. The pyrolysis oil composition of claim 1 suitable for use without further treatment in a refinery or a petrochemical process.

5. The pyrolysis oil composition of claim 1 wherein said pyrolysis oil composition is suitable for use without further treatment in combination with a refinery or a petrochemical process.

6. The pyrolysis oil composition of claim 1 wherein said trace elements are selected from calcium, silicon, aluminum and iron.

7. The pyrolysis oil composition of claim 1 wherein said halogens comprise chloride, fluoride and bromide containing molecules.

8. The pyrolysis oil composition of claim 1 comprising about 36-56 wt % olefins.

9. The pyrolysis oil composition of claim 1 comprising about 17-45 wt % paraffins.

10. The pyrolysis oil composition of claim 1 comprising about 14-20 wt % aromatics.

11. The pyrolysis oil composition of claim 1 comprising about 0.5-10 ppm by weight chloride.

12. The pyrolysis oil composition of claim 1 comprising about 0.5-10 ppm by weight of said total trace metal elements.

13. The pyrolysis oil composition of claim 1 derived from a recycled plastics composition comprising from about 0.5-10 wt % polyvinyl chloride.

14. The pyrolysis oil composition of claim 1 wherein said recycled plastics composition comprises about 0.2 to 2 wt % polyvinyl chloride.

15. A process of making a pyrolysis oil composition from a recycled plastics feed stream comprising sending said recycled plastics feed stream to a pyrolysis process to produce said pyrolysis oil composition comprising greater than 20 wt % olefins and between about 0.5 to 20 ppm halogens and a total trace element between about 0.5 to 10 ppm.

16. The process of claim 15 wherein said pyrolysis oil composition is suitable for use as a feed without further treatment in a refinery or a petrochemical reactor.

17. The process of claim 15 wherein said pyrolysis oil comprises about 36-56 wt % olefins about 17-40 wt % paraffins and about 14-20 wt % aromatics.

18. The process of claim 15 wherein said trace elements are selected from calcium, silicon, aluminum and iron.

19. The process of claim 15 wherein said halogens comprise chloride, fluoride and bromide containing molecules.

20. The process of claim 15 wherein said recycled plastics feed stream comprises about 0.2 to 10 wt % polyvinyl chloride.
